(19) United States
(12) Patent Application Publication
(10) Pub. No.: US 2022/0184198 A1
(43) Pub. Date: Jun. 16, 2022

(54) COMPOSITION FOR MUCOSAL ADMINISTRATION TO AVIANS

(71) Applicant: Phibro Animal Health Corporation, Teaneck, NJ (US)

(72) Inventors: Peter Winter, Omaha, NE (US); Karen Brown, Parkville, MO (US); Michelle Mathiesen, Omaha, NE (US); Hailong Guo, Shoreview, MN (US); Jeremy Griffel, Omaha, NE (US); Sam Christenberry, Omaha, NE (US); Meghan Quinn, Omaha, NE (US); Hector Cervantes, Omaha, NE (US); Bereket Zekarias, Omaha, NE (US)

(73) Assignee: Phibro Animal Health Corporation, Teaneck, NJ (US)

(21) Appl. No.: 17/582,200

(22) Filed: Jan. 24, 2022

Related U.S. Application Data
(63) Continuation of application No. PCT/US2020/043818, filed on Jul. 28, 2020.
(60) Provisional application No. 62/880,458, filed on Jul. 30, 2019.

(51) Int. Cl. A61K 39/08; A61K 39/112; A61P 31/04

(52) U.S. Cl. CPC A61K 39/08; A61K 2039/541; A61P 31/04; A61K 39/0275

(57) ABSTRACT

Inactivated antigens have only been used in avian medicine in parenterally administered vaccines. The only mucosally administered vaccines have been live, modified live, or attenuated vaccines. However, live vaccines have several disadvantages, including the risk of causing disease. Disclosed embodiments concern a composition comprising inactivated bacterial and/or viral antigens that is formulated for mucosal administration to an avian, and a method of using the composition. In certain embodiments, the composition comprises inactivated Clostridium perfringens type A antigens, and/or Salmonella spp. antigens, such as antigens from Salmonella kentucky, Salmonella typhimurium, and/or Salmonella enteriditis, or E. coli antigens. The composition may comprise a polyacrylic acid adjuvant. The composition may be administered to an avian in ovo, during the first 14 days after hatching, or after the first 14 days.

### CROSS REFERENCE TO RELATED APPLICATION

This application is a continuation of International Application No. PCT/US2020/043818, filed on Jul. 28, 2020, which was published in English under PCT Article 21(2), which in turn claims the benefit of the earlier filing date of U.S. provisional patent application No. 62/880,458, filed on Jul. 30, 2019, both of which are incorporated herein by reference in their entireties.

### FIELD

This disclosure relates to a composition comprising inactivated antigens and a mucosal adjuvant, for administration to avian species, and methods of making and using the composition.

### BACKGROUND

Inactivated vaccines have been used in avian medicine to help prevent diseases for many years. All such vaccines have been administered parenterally, such as by an intramuscular (IM) or subcutaneous (SC) route. The only vaccines administered mucosally to avian species, such as orally or as a spray, are those containing live, modified live or attenuated organisms (collectively defined herein as live vaccines). In these cases, the organisms are typically altered such that they should not produce significant active disease but may produce a mild syndrome that stimulates the immune system in a manner similar to the active diseases.

A disadvantage of the live vaccines is that they shed their organisms to the environment and/or to other animals and, in some cases allow the vaccine strain to recombine with field strains of the organisms circulating through populations of animals to produce devastating mutations (new viral or bacterial recombinants) that cause continued disease problems or even new epidemics.

### SUMMARY

Disclosed herein are embodiments of a composition comprising inactivated antigens and at least one mucosal adjuvant, where the composition is formulated for administration to an avian. The composition may comprise a polyacrylic acid mucosal adjuvant and/or inactivated antigens from respiratory or intestinal bacteria or viruses such as, but not limited to, Clostridium spp. such as Clostridium perfringens type A, Clostridium perfringens type C, Clostridium septicum, or Clostridium colinum; Haemophilus paragalinarum (Coryza); Escherichia spp. such as E. coli; Salmonella spp. such as Salmonella kentucky, Salmonella typhimurium, or Salmonella enteriditis; Pasteurella spp. such as Pasteurella multocida, or Pasteurella hemolyca; Staphylococcus spp.; Micrococcus spp.; Campylobacter spp. such as Campylobacter hepaticus, Campylobacter jejuni, or Campylobacter coli; Avibacterium spp.; Actinobacillus spp.; Neisseria spp.; Erysipelothrix spp.; Moraxella spp.; Avian Chlamydiosis (Chlamydia); Gallibacterium spp., such as Galibacterium anatis; Pseudomonas spp.; Rhodococcus spp.; Serratia spp.; Streptococcus spp.; Avian Mycoplasma species (Mycoplasma); Avian coccidiosis (Coccidia); reoviruses (REO); avian influenza viruses (IAV-A); Infectious bronchitis virus (IBV); Newcastle Disease Virus (NDV); Fowl Adenovirus (FA); Infectious bursal disease (IBD); Marek's Disease (MDV); Chicken Anemia (CAV); Infectious Larynogotracheitis (ILTV); Avian Encephalomyelitis (AEV); Avian hepatitis (HEV); Duck hepatitis (DHV); Turkey hemorrhagic enteritis (THEV); Egg Drop Syndrome virus (EDS); or a combination thereof.

In any embodiments, the composition may further comprise a vehicle, surfactant, inactivating agent, neutralizing agent, cell fragments, or a combination thereof. The inactivating agent may be formaldehyde, formalin, binary ethyleneimine, thimerosal, beta propiolactone, a detergent, or a combination thereof. However, in certain embodiments, the composition does not comprise saline solution.

The composition may have an osmolarity of from greater than zero to 2% (w/v) sodium chloride solution, and/or may have a substantially isotonic osmolarity. Additionally, or alternatively, the composition may have a viscosity of from greater than zero to 6 mPa·s, such as from 2 mPa·s to 5 mPa·s. And/or in some embodiments, the composition is formulated as a liquid or a suspension of particles within an aqueous base, but in other embodiments, the composition may be formulated as a gel. The gel may comprise a gelling agent, such as carboxymethyl cellulose, carboxymethyl chitosan, chitosan, sodium hyaluronate, polyethylene glycol, xantham gum, starches, pectins, gelatin, polysaccharides and oligopolysaccharides, carrageenan, derivatives and combinations thereof. Additionally, or alternatively, the composition may include added immunostimulants, such as a saponin, Quil A, dimethyldioctadecyl ammonium bromide, dimethyldioctadecyl ammonium chloride, poloxamer, polyethylene maleic anhydride, or a combination thereof.

The adjuvant may have an adjuvant concentration in the composition of from greater than zero to 80% (v/v), such as from 5% to 80%, or from greater than zero to 50%, from 0.5% to 50%, from 1% to 50% or from 5% to 50%. In some embodiments, the composition is formulated for addition to drinking water and the adjuvant concentration is from greater than zero to less than 100%, such as from 15% to 80%. Alternatively, the composition may be formulated for spray administration and have an adjuvant concentration of from greater than zero to 80%, such as from 0.5% to 80%, from 0.5% to 50%, from 1% to 50%, from 5% to 50% or from 15% to 40%. Or the composition may be formulated for administration to the eye and the adjuvant concentration may be from 5% to 25%. And in other embodiments, the composition is formulated for gel administration and the adjuvant concentration is from 15% to 40% prior to mixing with a gel composition, such as a composition comprising a gelling agent.

Also disclosed herein are embodiments of a drinking water composition comprising water and from greater than zero to less than 100% (v/v), such as from greater than zero to less than 80% (v/v), or from greater than zero to 50% (v/v) of the disclosed composition, and also embodiments of a gel composition, comprising a first composition comprising the disclosed composition, and a second composition comprising a gelling agent. The gel composition may have a ratio between the first composition to the second composition of from 25:75 to 75:25, such as 50:50.

Additional embodiments concern a method comprising administering a composition according to any of the disclosed embodiments to an avian. The method may comprise administering to an avian mucosal membrane. In some embodiments the avian is a chicken, turkey, goose, duck, Cornish game hen, quail, partridge, pheasant, guinea-fowl, ostrich, emu, swan, or pigeon, preferably a chicken or a turkey. And/or the composition may be delivered by any suitable route, such as orally, via the chloanal crest, via the harderian gland, ocularly or topically. Application may be made by spraying, misting, eye dropper, drinking water, feed and combinations thereof. Alternatively, the composition may be administered in ovo, such as by injection.

In certain embodiments, the composition is first administered to an avian of from greater than zero to 14 days of age, such as from 1 days to 9 days of age or to older avians, that may be used for breeding or laying, at any age. Additionally, in certain embodiments, the composition may be administered to the chick in ovo, such as by injecting the composition into the yolk, yolk sac and/or embryonic membrane.

Administering the composition may comprise a first administration of the composition followed by a second administration subsequent to the first administration. Or there may be just a single application or multiple re-administrations in long-lived avians. The first administration may occur when the avian is from greater than zero to 14 days of age. In some embodiments, the second administration is from greater than zero to 6 weeks after the first administration, such as from 1 day to 4 weeks after the first administration, or from 3 days to 10 days after the first administration. And in particular embodiments, the method may comprise administering a composition comprising inactivated antigens from Clostridium perfringens type A antigens, Salmonella spp., such as Salmonella kentucky, Salmonella typhimurium, and/or Salmonella enteriditis, and/or Escherichia spp., such as E. coli, and a polyacrylic acid adjuvant to an avian of from greater than zero to 14 days of age.

In any embodiments, administering to the avian may comprise spraying the composition onto the avian and/or administering the composition to the avian's eye. Alternatively, administering to the avian may comprise providing to the avian a drinking water composition or a gel composition that comprises the composition. The drinking water composition may comprise from greater than zero to less than 100% (v/v) of the composition, such as from 80% to less than 100%, or 70% to less than 100%. And in some embodiments, the method further comprises mixing the composition and water to form the drinking water composition.

A method of inducing an immune response in an avian species is also disclosed. The method may comprise administrating to the avian a composition according to any of the disclosed embodiments. Inducing an immune response may comprise inducing an IgA response and optionally both IgA and IgY responses. Also disclosed is a method of treating or preventing necrotic enteritis in an avian, where the method comprises administering to the avian a composition according to any of the disclosed embodiments, wherein the composition comprises inactivated Clostridium perfringens type A antigens. Another disclosure is a method of reducing the translocation of microorganisms such as Salmonella spp., and/or Escherichia spp., such as species that are related to human food safety issues cause by contaminated poultry meat. Such organisms may include, but are not limited to, Salmonella kentucky, Salmonella typhimurium, Salmonella enteriditis, and Escherichia coli.

Additionally, disclosed herein is a method comprising mucosally administering a composition comprising inactivated antigens and at least one mucosal adjuvant to an avian. Administering the composition may comprise spraying the composition on to the avian, and/or may comprise administering the composition ocularly, nasally and/or orally. The composition may comprise from 80% to less than 100% water and/or comprise an aqueous-based adjuvant. In some embodiments, the composition comprises from 0.5% to 50% adjuvant, such as from 1% to 50% adjuvant or from 5% to 50% adjuvant, and/or the adjuvant may be a polyacrylic acid adjuvant.

In some embodiments, the composition is a suspension, and may be an aqueous suspension comprising polyacrylic acid particles, such as polyacrylic acid particles having particle size of from 250 nm to 10 microns.

The inactivated antigens may comprise antigens from, but not limited to, Clostridium spp. such as Clostridium perfringens type A, Clostridium perfringens type C, Clostridium septicum, or Clostridium colinum; Haemophilus paragalinarum (Coryza); Escherichia spp. such as E. coli.; Salmonella spp. such as Salmonella kentucky, Salmonella typhimurium, or Salmonella enteriditis; Pasteurella spp. such as Pasteurella multocida, or Pasteurella hemolyca; Staphylococcus spp.; Micrococcus spp.; Campylobacter spp. such as Campylobacter hepaticus, Campylobacter jejuni, or Campylobacter coli; Avibacterium spp.; Actinobacillus spp.; Neisseria spp.; Erysipelothrix spp.; Moraxella spp.; Avian Chlamydiosis (Chlamydia); Gallibacterium spp., such as Galibacterium anatis; Pseudomonas spp.; Rhodococcus spp.; Serratia spp.; Streptococcus spp.; Avian Mycoplasma species (Mycoplasma); Avian coccidiosis (Coccidia); reoviruses (REO); avian influenza viruses (IAV-A); Infectious bronchitis virus (IBV); Newcastle Disease Virus (NDV); Fowl Adenovirus (FA); Infectious bursal disease (IBD); Marek's Disease (MDV); Chicken Anemia (CAV); Infectious Larynogotracheitis (ILTV); Avian Encephalomyelitis (AEV); Avian hepatitis (HEV); Duck hepatitis (DHV); Turkey hemorrhagic enteritis (THEV); Egg Drop Syndrome virus (EDS); or a combination thereof.

The composition may further comprise cell fragments, an inactivating agent, a surfactant, neutralizing agent, or a combination thereof, and the inactivating agent may be formaldehyde, formalin, binary ethyleneimine, thimerosal, beta propiolactone, a detergent, or a combination thereof. Additionally, or alternatively, the composition may have a viscosity is from 2 mPa·s to 5 mPa·s; an isotonic osmolarity; a pH of from 6.5 to 7.5; or a combination thereof. In some embodiments, the avian is a chicken, turkey, goose, duck, Cornish game hen, quail, partridge, pheasant, guinea-fowl, ostrich, emu, swan, or pigeon, and in certain embodiments, the avian is a chicken or turkey.

The composition may be administered to the avian on from day of hatch to day 14, such as on from day of hatch to day 3, and in certain embodiments, the composition is administered to the avian on day of hatch.

In some embodiments, administering the composition comprises administering a first composition comprising inactivated antigens and at least one mucosal adjuvant, and the method further comprises subsequently administering a second composition to the avian. The second composition may be administered from greater than zero to 6 weeks after the first composition is administered, such as from 5 days to 20 days, or from 10 days to 15 days after the first composition is administered. The second composition may comprise the same inactivated antigens and mucosal adjuvant as the first composition, or alternatively, the second composition may comprise different inactivated antigens and/or a different mucosal adjuvant from the first composition. In some embodiments, the second composition is administered mucosally, such as by spraying and/or orally, but in alternative embodiments, the second composition is administered by injection using a non-mucosal adjuvant.

In any embodiments, the method may be a method for reducing the incidence of Salmonella spp., such as when the Salmonella spp. is S. kentucky. S. enteriditis or S typhimurium, an avian or in meat obtained from the avian after harvest.

In any embodiments, the method may be a method for reducing the incidence of Escherichia spp., such as when the Escherichia spp. is Escherichia coli, in an avian or in meat obtained from the avian after harvest.

In a particular embodiment, the method comprising spraying onto the avian, an aqueous suspension comprising inactivated antigens, polyacrylic acid particles, and from 70% to less than 100% water. And the method may comprise spraying onto the avian, an aqueous suspension comprising inactivated antigens selected from antigens from one or more of Clostridium spp. (such as Clostridium perfringens type A, Clostridium perfringens type C, Clostridium septicum, or Clostridium colinum), Haemophilus paragalinarum (Coryza), Escherichia spp. (such as E. coli.), Salmonella spp. (such as Salmonella kentucky, Salmonella typhimurium, or Salmonella enteriditis), Pasteurella spp. (such as Pasteurella multocida, or Pasteurella hemolyca), Staphylococcus spp., Micrococcus spp., Campylobacter spp. (such as Campylobacter hepaticus, Campylobacter jejuni, or Campylobacter coli), Avibacterium spp., Actinobacillus spp., Neisseria spp., Erysipelothrix spp., Moraxella spp., Avian Chlamydiosis (Chlamydia), Gallibacterium spp. (such as Galibacterium anatis), Pseudomonas spp., Rhodococcus spp., Serratia spp., Streptococcus spp., Avian Mycoplasma species (Mycoplasma), Avian coccidiosis (Coccidia), reoviruses (REO), avian influenza viruses (IAV-A), Infectious bronchitis virus (IBV), Newcastle Disease Virus (NDV), Fowl Adenovirus (FA), Infectious bursal disease (IBD), Marek's Disease (MDV), Chicken Anemia (CAV), Infectious Larynogotracheitis (ILTV), Avian Encephalomyelitis (AEV), Avian hepatitis (HEV), Duck hepatitis (DHV), Turkey hemorrhagic enteritis (THEV), or Egg Drop Syndrome virus

(EDS); polyacrylic acid particles having a particle size of from 250 nm to 10 microns; cell fragments, an inactivating agent, a surfactant, neutralizing agent, or a combination thereof; and from 80% to less than 100% water.

Also disclosed is a composition comprising inactivated antigens and at least one mucosal adjuvant for use in a method comprising administering the composition to an avian.

The foregoing and other objects, features, and advantages of the invention will become more apparent from the following detailed description, which proceeds with reference to the accompanying figures.

### BRIEF DESCRIPTION OF THE DRAWINGS

FIG. 1 is a graph of percent mortality versus treatment group, illustrating the percentage mortality due to necrotic enteritis for each treatment group in the study.

FIG. 2 is a graph of average necrotic enteritis lesion scores versus treatment, illustrating the average necrotic enteritis intestinal lesion scores on day 21 for each treatment group.

### DETAILED DESCRIPTION

### I. Definitions

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

The term “adjuvant” refers to any component added to a vaccine that enhances the immune response. The term includes any adjuvant suitable for administration to an avian subject. In some embodiments, the adjuvant is, or comprises, CARBIGEN™; maleic anhydrides, such as ethylene maleic anhydride or polyethylene maleic anhydride; aluminum salts, such as aluminum hydroxide or aluminum phosphate; EMULSIGEN -based adjuvants including EMULSIGEN®, EMULSIGEN®-D (containing dimethyldioctadecylammonium bromide (DDA)), EMULSIGEN®-BCL (containing a block copolymer immunostimulant), and EMULSIGEN®-P (containing with a proprietary immunostimulant) (Phibro Animal Health Corporation, Omaha, Neb., USA); water-in-oil emulsions; oil-in-water emulsions; saponins including VetSap, QS21 (Antigenics, Framingham, Mass.) and derivatives of saponins; polyacrylic acids; copolymers, including low molecular weight copolymers such as Polygen™ (available from Phibro Animal Health Corporation, Omaha, Neb., USA); Carbopol-based adjuvants; synthetic polynucleotides such as oligonucleotides containing a CpG motif (e.g., U.S. Pat. No. 6,207,646); TRIGEN™; Poly-IC; Poly-ICLC; carboxymethyl chitosan, chitosan, MONTANIDES; or combinations thereof.

Bacterial antigens suitable for use in the present technology include proteins, polysaccharides, lipopolysaccharides, and/or outer membrane vesicles which may be purified, isolated or derived from a bacterium. Bacterial antigens also may include bacterial lysates and inactivated bacteria formulations. In some embodiments, bacteria antigens may be produced by recombinant expression. Typically, bacterial antigens include epitopes which are exposed on the surface of the bacteria during at least one stage of a life cycle. Bacterial antigens may be conserved across multiple serotypes. Bacterial antigens include antigens derived from one or more of the bacteria disclosed herein.

Viral antigens suitable for use in the present technology include inactivated (or killed) virus and/or viral proteins which may be isolated, purified or derived from a virus. Viral antigens can be derived from viruses propagated on a substrate, such as a cell culture or other substrate, or they may be derived or expressed recombinantly, viral antigens including epitopes which are exposed on the surface of the virus during at least one stage of a life cycle or antigenic peptides that are purified from viruses or synthetically produced. Viral antigens may be conserved across multiple serotypes or isolates. Viral antigens include antigens derived from one or more of the viruses disclosed herein. Viral antigens of interest may include antigens from respiratory or intestinal viruses disclosed herein including, but not limited to, reoviruses (REO), avian influenza viruses (IAV-A), Infectious bronchitis virus (IBV), Newcastle Disease Virus (NDV), Fowl Adenovirus (FA) and Egg Drop Syndrome virus (EDS).

The terms “mucoadhesive,” “mucosal adjuvant,” or “mucosally-adjuvanted” refer to an adjuvant that has the capability to adhere to mucosal membranes and stimulate an immune response. Mucous membranes include the nasopharyngeal, oral, optic (eye), vaginal or anal membranes. The immune response that is stimulated may include IgA, IgY, IgM, or a combination thereof, which are found in the serum and in mucosal washings. Compositions comprising such adjuvants are administered to the mucosal membranes of animals.

The term “live vaccine” refers to a vaccine comprising live, modified live, and/or attenuated organisms, as opposed to inactivated antigens or killed organisms. The term “inactivated antigens” refers to antigens that are unable to reproduce and/or cause disease. Inactivated antigens include, but are not limited to, antigens from killed organisms, subunits, recombinant antigens, or combinations thereof. An “inactivated vaccine” is a vaccine that comprises inactivated antigens, and does not include live, modified live and/or attenuated organisms.

As used herein, administering a vaccine mucosally includes both directly administering the vaccine to the avian, such as by directly administering the vaccine to the avian's mouth, eye, harderian gland, choanal cleft or anus, and providing the vaccine such that the avian ingests the vaccine, such as providing a vaccine for the birds to eat, drink or peck off of each other. Exemplary methods of providing the vaccine include, but are not limited to, spraying the vaccine on the birds, and/or otherwise applying the vaccine to the feathers, such that the vaccine is ingested as the birds peck at each other's feathers, and/or preen themselves, or providing the vaccine in a form that the birds will ingest, such as in a gel that birds will peck at, or a liquid that the birds will drink. A person of ordinary skill in the art will understand that spraying may also facilitate direct administration because spray droplets may directly enter the mouth, choanal cleft and/or eye of a bird. Spraying may be performed by any suitable technique, such as a backpack sprayer or a spray cabinet. Typically, a suitable spraying technique will spray the composition onto the upper exposed surfaces of the bird as possible, such as the bird's back, head, face, tail feathers, etc.

Infection or challenge means that the subject has been exposed to live disease-causing organisms that may result in the subject exhibiting one or more clinical signs of the disease.

The terms “effective amount” or “therapeutically effective amount” refer to the amount of an active agent (such as one or more embodiments provided herein alone, in combination, or potentially in combination with other therapeutic agent(s)) sufficient to induce a desired biological result. That result may be reduction in disease signs, amelioration or alleviation of the signs, symptoms, or causes of a disease, or any other desired alteration of a biological system. The term “effective amount” or “therapeutically effective amount” is used herein to denote any amount of a therapeutic and/or preventative that causes an improvement in a disease condition, or prevention of disease symptoms. The amount can vary with the condition being treated, the stage of advancement of the condition, the type and concentration of formulation applied, and the age, sex and species of the subject. Appropriate amounts in any given instance will be readily apparent to those of ordinary skill in the art or capable of determination by routine experimentation such as vaccination and observation of an antibody response or vaccination followed by a challenge wherein the vaccinated animals perform better than non-vaccinated animals that are challenged similarly.

The term “gel” refers to a colloidal system comprising a solid three-dimensional network within a liquid. By weight, a gel is primarily liquid, but behaves like a solid due to a three-dimensional network of entangled and/or crosslinked molecules of a solid within the liquid. From a rheological perspective, a gel has a storage modulus G′ value which exceeds that of the loss modulus G″. The storage modulus is a measure of the energy stored in a material in which a deformation (e.g., sinusoidal oscillatory shear) has been imposed; storage modulus can be thought of as the proportion of total rigidity of a material that is attributable to elastic deformation. The loss modulus is a measure of the energy dissipated in a material in which a deformation (e.g., sinusoidal oscillatory shear) has been imposed; loss modulus can be thought of as the proportion of the total rigidity of a material that is attributable to viscous flow rather than elastic deformation. The storage modulus and loss modulus can be determined with a rheometer.

A gel may disperse when added to water. However, when applied to the feathers of a bird, the gel typically will stick to the feathers so that the bird to which it is applied, and/or other birds, can peck the gel off the feathers and thus the disclosed antigen composition that is contained within the gel is administered to the bird(s). A gel may be formed by including a gelling agent in the disclosed composition. Suitable gelling agents may include carboxymethyl cellulose, carboxymethyl chitosan, chitosan, sodium hyaluronate, polyethylene glycol, xantham gum, starches, pectins, gelatin, polysaccharides and oligopolysaccharides, carrageenan, derivatives and combinations thereof

### II. Overview

Currently, there are no avian vaccines comprising inactivated antigens that are administered mucosally. However, inactivated vaccines have certain advantages over live vaccines, including reducing the risk of shedding the organism and/or recombining with field strains to form new variants of the disease. Additionally, mucosal administration has several advantages over parental administration. For example, spray administration removes the need to individually handle multiple birds, thereby substantially reducing the cost of administering the vaccine to a group of birds by reducing the associated workload. And mucosal administration also results in the avian not being exposed to potentially stressful needle injections, reduces the potential of meat loss due to the presence of residual adjuvant/vaccine at the injection site, and also reduces or substantially eliminates the risk of accidental self-injection by a worker. Additionally, because mucosal administration produces a significant IgA response, it is more probable that such a route of administration of inactivated antigens overcomes the maternal antibody that is obtained from the hen passed in ovo.

One exemplary avian disease for which the disclosed method and composition is useful is necrotic enteritis. The disease is caused by Clostridium perfringens type A and produces a severe gastroenteritis with intestinal lesions, weight loss and death in juvenile poultry, typically within the first 10 to 15 days of age. An additional issue occurs at slaughterhouses when infected birds are harvested. The process line must close down and extensive cleanup must occur at a significant cost to the slaughterhouse if lesions are found and there is a potential of infecting the premises with C. perfringens. No vaccine has been found to protect poultry against this disease. And all vaccines that have been tried have been administered parenterally.

The ideal approach is to be able to administer inactivated vaccines that replicate the protection observed with live vaccines. The inventors surprisingly discovered a composition comprising inactivated antigens that can be administered mucosally, such as orally, ocularly or topically, to protect animals from disease as demonstrated in vaccination/challenge tests using Clostridium perfringens type A.

Another exemplary use of an inactivated vaccine that can be applied mucosally, is to reduce Salmonella spp. and/or Escherichia spp. in poultry. These organisms may not cause disease in the poultry but they can be shed to humans via the meat, causing food born illnesses that can lead to severe gastroenteritis and, in some cases, death. These bacteria are normally found in the intestinal tract of poultry. They are known to translocate to organs and even to the meat of birds. The inventors have surprisingly discovered that levels of Salmonella spp. and Eschericia spp. can be reduced in poultry, thereby potentially improving the safety of poultry meat for human consumption.

### III. Composition

Disclosed herein are embodiments of a composition comprising inactivated antigens and a mucosal adjuvant, such as mucoadhesive adjuvant. The inactivated antigens may be any suitable antigen, such as antigens from a bacterial and/or viral avian disease. The composition may be formulated for mucosal administration to avians, such as oral, nasal, ocular, vaginal, or anal administration, or a combination thereof. The composition may stimulate an immune response in the avian, thereby helping to reduce the severity and incidence of disease when the subject is later challenged by exposure to live organisms. The immune response may comprise an IgA immune response and also an IgY response.

Exemplary antigens suitable for use in the disclosed composition include inactivated antigens from any suitable avian bacterial or viral disease. In some embodiments, the composition comprises antigens obtained from one or more bacteria and/or virus, such as from 1, 2, 3, 4, 5, 6, 7, 8, 9, 10 or more bacteria and/or viruses. In certain disclosed embodiments, the composition comprises inactivated antigens obtained from one or more of the following organisms: Clostridium spp. such as Clostridium perfringens type A, Clostridium perfringens type C, Clostridium septicum, or Clostridium colinum; Haemophilus paragalinarum (Coryza); Escherichia spp. such as E. coli; Salmonella spp. such as Salmonella kentucky, Salmonella typhimurium, or Salmonella enteriditis; Pasteurella spp. such as Pasteurella multocida, or Pasteurella hemolyca; Staphylococcus spp.; Micrococcus spp.; Campylobacter spp. such as Campylobacter hepaticus, Campylobacter jejuni, or Campylobacter coli; Avibacterium spp.; Actinobacillus spp.; Neisseria spp.; Erysipelothrix spp.; Moraxella spp.; Avian Chlamydiosis (Chlamydia); Gallibacterium spp., such as Galibacterium anatis; Pseudomonas spp.; Rhodococcus spp.; Serratia spp.; Streptococcus spp.; Avian Mycoplasma species (Mycoplasma); Avian coccidiosis (Coccidia); reoviruses (REO); avian influenza viruses (IAV-A); Infectious bronchitis virus (IBV); Newcastle Disease Virus (NDV); Fowl Adenovirus (FA); Infectious bursal disease (IBD); Marek's Disease (MDV); Chicken Anemia (CAV); Infectious Larynogotracheitis (ILTV); Avian Encephalomyelitis (AEV); Avian hepatitis (HEV); Duck hepatitis (DHV); Turkey hemorrhagic enteritis (THEV); or Egg Drop Syndrome virus (EDS).

In some embodiments, the inactivated antigens include, but are not limited to, whole culture bacteria; whole culture virus; subunits obtained from bacteria or virus that have been extracted or separated from the culture; subunits that have been extracted or separated from the cells; antigens obtained from recombinant organisms other than the particular bacteria or virus(es) of primary interest, but which protect against infection or challenge by the bacteria or virus(es) of primary interest; or combinations thereof In some embodiments, the bacteria or virus(es) of primary interest include, but are not limited to, Clostridium perfringens type A, E. coli, and/or Salmonella spp., such as Salmonella kentucky, Salmonella typhimurium, or Salmonella enteriditis. Such antigens may also be combined with other antigens that are typically administered to avians. Such additional antigens include, but are not limited to, antigens from bacteria and/or viruses disclosed herein.

The composition may comprise the inactivated antigens and a suitable adjuvant, such as a mucosal, or mucoadhesieve, adjuvant. In some embodiments, the mucosal adjuvant is CARBIGEN™; maleic anhydrides, such as ethylene maleic anhydride or polyethylene maleic anhydride; aluminum salts, such as aluminum hydroxide or aluminum phosphate; EMULSIGEN®-based adjuvants including EMULSIGEN®, EMULSIGEN®-D (containing dimethyldioctadecylammonium bromide (DDA)), EMULSIGEN®-BCL (containing a block copolymer immunostimulant), and EMULSIGEN®-P (containing with a proprietary immunostimulant) (Phibro Animal Health Corporation, Omaha, Neb., USA); chitosan, hydroxymethyl chitosan, water-in-oil emulsions; oil-in-water emulsions; saponins including VetSap, QS21 (Antigenics, Framingham, Mass.) and derivatives of saponins; polyacrylic acids; copolymers, including low molecular weight copolymers such as Polygen™ (available from Phibro Animal Health Corporation, Omaha, Neb., USA); Carbopol-based adjuvants; synthetic polynucleotides such as oligonucleotides containing a CpG motif (e.g., U.S. Pat. No. 6,207,646); TRIGEN™; Poly-IC; Poly-ICLC; MONTANIDES; or combinations thereof. In certain embodiments, the adjuvant is CARBIGEN™, a polyacrylic acid, saponin, POLYGEN™, ethylene maleic anhydride, ENABL®, or a combination thereof. In a preferred embodiment, the adjuvant is at a concentration in the composition of from greater than zero to 80% or more (v/v), such as from 0.5% to 80%, from 1% to 80%, from 5% to 80% from 10% to 80%, from 10% to 70%, from 10% to 60% or from 10% to 50%. In some embodiments, such as certain embodiments formulated for administration to the eye, the adjuvant concentration may be from 0.5% to 25% (v/v) or more, such as from 5% to 25%, such as from 10% to 20%. In other embodiments, such as certain embodiments formulated for gel administration, the adjuvant concentration may be from 15% to 40% (v/v) or more, such as from 15% to 35% or from 20% to 30%. For spray formulations, the adjuvant may have a concentration of from 0.5% to 80% (v/v) or more, such as from 0.5% to 50%, from 1% to 50%, from 5% to 50% or from 10% to 50%. And in other certain embodiments, such as embodiments formulated for addition to drinking water, the adjuvant concentration may be from 0.5% to 80%, (v/v) or more, such as from 15% to 80%, or from 20% to 80%. In such embodiments, the resulting composition may be added to water at a concentration of from greater than zero to 20% (v/v) or more, such as from 5% to 15% or at 10% (v/v) in the water to form a drinking water composition. The drinking water composition may also comprise a colored dye that can indicate whether a particular bird has drunk the water by staining the beak.

In some embodiments, the composition may further comprise a vehicle, preservative, neutralizing agent, surfactant, inactivating agent, cell fragments, media, or any combination thereof. The vehicle may be any suitable vehicle that does not cause irritation to the respective mucosa. The vehicle may be a diluent, such as water, aqueous saline, phosphate buffered saline (PBS), culture media, or a combination thereof, or other known substances that can be used in combination with other components of disclosed compositions.

The preservative may be any preservative suitable for use in the disclosed composition. Exemplary preservatives include, but are not limited to, gentamicin, penicillin, streptomycin, polymyxin B, formaldehyde, formalin, thimerosal, or combinations thereof. Inactivating agents suitable for use in the disclosed composition include, but are not limited to, formaldehyde, formalin, binary ethyleneimine, thimerosal, beta propiolactone, detergents such as NP40 and Triton X 100, and combinations thereof. Additionally, or alternatively, the disclosed composition may comprise a neutralizing agent, such as sodium thiosulfate and/or sodium bisulfite. And/or a surfactant may be present and may enhance absorption of the subject proteins by the nasal mucosa. Suitable surfactants include, but are not limited to, tweens, spans and detergents such as NP40 and Triton X 100, or any combination thereof. If present in the composition, a surfactant, such as a detergent, may be present in an amount of from greater than zero to less than 100%, such as from greater than zero to 50%, from greater than zero to 35% or from greater than zero to 25%. However, certain embodiments of the composition do not comprise a surfactant.

And the cell fragments and/or media may comprise fragments of the cells formed when the antigens are inactivated and/or the media that the bacteria or virus was cultured to product the antigens. If present in the composition, the cell fragments and/or media may be present in an amount of from greater than zero to less than 100%, such as from greater than zero to 75%, from greater than zero to 65% or from greater than zero to 50%. However, certain embodiments of the composition do not comprise cell fragments and/or media.

In a particular embodiment, the composition comprises inactivated antigens, such as antigens from C. perfringens Type A, Salmonella kentucky, Salmonella typhimurium, Salmonella enteriditis, and/or E. coli, a polyacrylic acid adjuvant, such as Carbigen™, formaldehyde, and optionally, cell fragments and/or growth media.

In some embodiments, the composition does not comprise saline solution, including PBS and/or saline solution or PBS is not added to the composition. However, a person of ordinary skill in the art will understand that certain embodiments of the composition may comprise a residual amount of saline if saline is present in the growth media that is used.

In some embodiments, the composition is not an emulsion, such as not a nanoemulsion, and/or not an oil-based emulsion.

In some embodiments, the composition has an osmolarity of from greater than zero to 2% (w/v) sodium chloride solution, such as from 0.1% to 1.9%, from 0.2% to 1.8%, from 0.3% to 1.7%, from 0.4% to 1.6%, from 0.4% to 1.5%, from 0.4% to 1.4%, from 0.4% to 1.3%, from 0.5% to 1.2%, or from 0.5% to 1.1%, or from 0.5% to 1% (w/v) sodium chloride solution. In certain embodiments, the composition has an osmolarity substantially the same as that of an avian tissue cell, typically about the osmolarity of a 0.5-0.9% (w/v) sodium chloride solution. In certain embodiments, the composition has a substantially isotonic osmolarity, and may be from 300 to 312 mOsm/L.

In some embodiments, the composition has a pH of from 6.5 to 7.5.

In some embodiments, the composition has a viscosity of from greater than zero to 6 mPa·s or more, such as from greater than zero to 5 mPa·s, from 1 mPa·s to 5 mPa·s, or from 2 mPa·s to 5 mPa·s. In some embodiments, the viscosity is less than 5 mPa·s, such as from greater than zero to less than 5 mPa·s, from 1 mPa·s to less than 5 mPa·s, or from 2 mPa·s to less than 5 mPa·s. A person of ordinary skill in the art will understand that when the disclosed composition is formulated as a gel, such as for birds to peck at as described herein, the gel composition will have a substantially greater viscosity than that of a liquid formulation, such as a spray or eye drop formulation. And in embodiments formulated for addition to drinking water, the viscosity of the resulting drinking water composition typically will have a lower viscosity than that of the adjuvanted immunogenic composition before it is added to the drinking water.

In some embodiments, the composition is used in an autogeneous inactivated vaccine. An autogeneous vaccine is produced from bacterial or viral strains that are isolated from a biological sample obtained from an animal in an infected herd. As such, an autogeneous vaccine often is strain specific, and therefore may produce better results that a generic over-the-counter vaccine. An autogeneous vaccine may only be available to the veterinarian that supplied the biological sample, but the product also may be made available to adjacent or at-risk businesses in the same region if there is a risk of the disease spreading if they use the same veterinarian.

Alternatively, the composition may be used in a fully-licensed vaccine that can be sold anywhere in the U.S. as well as in other countries where it meets the regulatory requirements.

In a particular embodiment, the composition comprises from 80% to less than 100% water, such as comprising at least 85% water, at least 90% water or at least 95% water. The composition may comprise an aqueous-based adjuvant, such as an aqueous-based adjuvant comprising a polyacrylic acid. A person of ordinary skill in the art understands that if such compositions comprise, for example, from 0.5% to 50% adjuvant, such as from 1% to 50%, from 5% to 50%, from 10% to 50%, or from 15% to 50% adjuvant, the aqueous-based adjuvant also comprises water, and so the composition may comprise both up to 50% adjuvant and up to 95% water.

In certain embodiments, an aqueous-based adjuvant is a polyacrylic acid-based adjuvant. In some embodiments, such the polyacrylic acid may form a suspension in the water The suspension may comprise polyacrylic acid particles having a size of from greater than 250 nm to 10 microns or more, such as from 300 nm to 10 microns, from 500 nm to 10 microns, from 1 micron to 10 microns, from 1 micron to 5 microns, or from 1 micron to 3 microns.

In any embodiments, the composition may be diluted by an end user, such as by water, to make a final composition suitable for administration by a desired route. In some embodiments, the desired route is spraying, and making the final composition may comprise diluting the composition with water, to form a water:composition ratio of from greater than zero:1 to 2 or more:1 water:composition, such as from 0.5:1 to 1.5:1 water:composition, or about 1:1 water:composition.

### IV. Administration

In some embodiments, the composition is administered mucosally, such as orally, topically or ocularly, to an avian. Exemplary avians include, but are not limited to, chickens, including laying hens, breeders and broilers, turkey, goose, duck, Cornish game hen, quail, partridge, pheasant, guinea-fowl, ostrich, emu, swan, or pigeon. In certain embodiments the avian is a chicken or turkey. The composition may be administered to an avian of any age. In some embodiments, the initial administration is to avians of from day of hatching to 14 days of age, such as from day of hatching to 10 days, from day of hatching to 5 days, or from day of hatching to 3 days. The composition may be administered at day 0, 1, 2, 3, 4, 5, 6, 7, 8, 9, or 10, and in some embodiments, the composition is administered at the day of hatching, for example, by spraying. It may also be administered up to within 7 days of processing the carcass. In addition, it may be administered daily in the drinking water throughout the life of the avian.

Administering the composition mucosally to the avian typically results in an immune response, such as an IgA immune response and/or an IgY response. The immune response may help to overcome maternal antibodies that the avian may receive from a hen that is positive for a bacteria or virus. Such maternal antibodies may be passed to the embryo in ovo. If the avian receives maternal antibodies while in ovo and then receives a parenteral vaccination after hatching, the maternal antibodies often block the subject from developing its own protective antibodies. The method of mucosal vaccination, such as the administration routes described herein, may produce an IgA immune response that can help overcome the effect of the maternal antibodies transferred to the bird.

Immunogenic compositions for mucosal administration, such as oral, topical or ocular administration, have several advantages over compositions that are administered by other routes, such as the intramuscular or subcutaneous routes. The advantages include, but are not limited to: 1) protecting the juvenile birds using humane techniques; 2) not exposing juvenile birds to stressful needle injections; 3) not involving injecting birds with live or modified live organisms that can shed and spread disease; 4) allowing the juvenile birds to develop IgA antibodies that can overcome the effect of any maternal IgY antibodies transferred from the parent; 5) not leaving any injection site lesions and thus allowing a zero day withdrawal time; 6) being easier to administer and reducing the workload; 7) reducing or substantially eliminating the risk of accidental self-injection of the worker; and/or 8) being able to be administered in the face of an outbreak to stop disease spread. Other advantages may be apparent from the description and the example section below.

In some embodiments, the disclosed composition is formulated for mucosal administration to avians. Mucosal administration includes, but is not limited to, oral, ocular, nasal, topical, and/or anal administration. In some embodiments, the composition is delivered ocularly, such as by eye drops or by spray. In other embodiments, the composition is administered orally, and may be administered by spraying the bird or providing a gel comprising the composition such that the birds peck at and thereby eat the gel/composition mixture. In some embodiments, the gel is administered topically to the back of the birds, such that other birds peck at the gel spot. The gel may be colored to attract pecking, such as with a red or blue color. In such embodiments, the adjuvanted immunogenic composition is mixed with a gelling composition comprising a gelling agent in a ratio suitable to provide an effective amount of the adjuvanted immunogenic composition to the bird(s). The ratio may be from 25:75 (immunogenic composition:gelling composition) to 75:25, such as from 34:66 to 66:34, or from 40:60 to 60:40, and in some embodiments, the ratio is 50:50.

Alternatively, the composition may be provided as a liquid or suspension for the birds to ingest. The liquid or suspension may be provided as a drinking liquid, or it may be sprayed as a liquid or suspension onto the birds. In some embodiments, spraying the birds may administer the composition ocularly, nasally, topically, and/or orally, either directly to the eye, nasal cavity, chloanal crest, and/or mouth, or indirectly, such as by birds ingesting the composition as they peck at each other and/or preen their feathers. In certain embodiments, spraying the composition administers the composition ocularly, nasally and optionally orally.

In some embodiments, a dose of the composition suitable for mucosal administration is from 0.15 mL or less per bird to 0.35 mL or more per bird, such as from 0.2 mL per bird to 0.3 mL per bird, or about 0.25 mL per bird. When administered mucosally, for example, by spraying or providing in drinking water, the composition may be administered in an amount of from 15 mL or more/100 birds to 35 mL or more/100 birds, such as from 20 mL/100 birds to 30 mL/100 birds, or about 25 mL/100 birds. In some embodiments, each bird theoretically receives about 80% of a dose, such as a 0.25 mL dose, of the composition.

Some embodiments of the disclosed composition are able to induce protective levels of antibodies as measured by the ELISA in >50%, typically >80%, >85%, >90%, or >95% of the individuals that are administered the composition. In some embodiments, the mucoadhesive composition also is able to maintain protective levels of antibodies against strains of bacteria and/or viruses, such as Clostridium perfringens type A, Salmonella kentucky, Salmonella typhimurium, Salmonella enteriditis and/or E. coli, throughout the early growth phase of an avian after hatching.

Thus, in certain embodiments, the composition can produce a persistent immune response against Clostridium perfringens type A, Salmonella kentucky, Salmonella typhimurium, Salmonella enteriditis and/or E. coli, and/or other avian diseases, such as the diseases listed herein. As used herein, a “persistent immune response” refers to a protective antibody immune response which is capable of protecting avians throughout their growth period from hatching to adult.

Embodiments of the immunogenic composition that comprise inactivated antigens from one or more strains of an avian disease, such as a diseases disclosed herein, for example, Clostridium perfringens type A, Salmonella kentucky, Salmonella typhimurium, Salmonella enteriditis and/or E. coli, where each strain independently is grown to titers greater than 10² CFU/mL, preferably greater than 10⁵ CFU/mL, and more preferably greater than 10⁷ CFU/mL, induce high titers of IgA antibodies in chicks.

In some embodiments, the disclosed composition is an inactivated vaccine.

The avians may receive a single dose of the disclosed composition, but in alternative embodiments, one or more additional administrations of a composition comprising one or more inactivated antigens obtained from one or more avian diseases, such as one or more strains of Clostridium perfringens type A, Salmonella kentucky, Salmonella typhimurium, Salmonella enteriditis and/or E. coli, may be necessary after the initial administration of the disclosed composition. The second administration may be from greater than zero to 6 weeks or more after the initial administration, such as from 1 day to 6 weeks, from 1 day to 5 weeks, from 1 day to 4 weeks, from 5 days to 3 weeks, from 5 days to 20 days, from 10 days to 20 days, or from 10 days to 15 days after the initial administration. A second administration and/or multiple booster administrations may also be given throughout the life of the avian that is used for breeding, laying or even as a pet. This second administration, and any subsequent administrations, also may be delivered mucosally, such as by spraying, or alternatively, it may be delivered parenterally and may include mucosal and/or non-mucoadhesive adjuvants. For instance, the second and/or additional administrations may be mucosal, such as oral, topical or ocular; intramuscular; subcutaneous; intraperitoneal; intravenous; or a combination thereof In certain embodiments, the disclosed composition is administered at least twice and both the first and the second administrations are via mucosal routes of administration, such as by spraying. But in other embodiments, the first administration is mucosal but the second and/or subsequent administration(s) is a by non-mucosal route.

In some embodiments, the composition administered by the second and/or subsequent administrations is the same as the composition that is initially administered, such as comprising the same antigens and adjuvant in substantially the same concentrations. But in other embodiments, the second and/or subsequent administrations comprise administering a composition that is different from the initial administration, even if both or all administrations are by the same route of administration. For example, a composition administered as a second and/or subsequent administration may comprise different antigens and/or a different adjuvant, and/or the concentration of antigens and/or adjuvant may be different from those in the initial composition.

### V. Examples

### Example 1

Necrotic enteritis is a severe gastrointestinal disease of young chickens that is caused by Clostridium perfringens type A. Currently, there are no commercially available vaccines to prevent or treat this disease. The focus of the study was to determine if various methods of vaccination using a killed antigen adjuvanted with a mucoadhesive adjuvant could provide protection.

### A. Growth of the C. perfringens

Several intestinal tissue samples were obtained from a producer who was experiencing deaths caused by C. perfringes type A. The intestinal contents were placed onto blood agar plates specific for the isolation of anaerobes. Five isolates were obtained. Each isolate was then streaked onto lake-sheep blood agar in flasks that were incubated under anaerobic conditions and then inoculated into modified tryptic soy broth containing 0.1% dextrose and 1% zinc sulfate. After growth was obtained, each grown isolate was analyzed for the production of Alpha toxin, Beta 2 toxin and Neuraminidase, as well as the TpeLsc and NetB genes using PCR. Table 1 provides the results of this testing. Intestinal sample 4 produced the highest alpha toxin, and accordingly, was chosen for vaccine production. The isolate seed was inoculated into tryptic soy broth supplemented with 0.1% dextrose and 1% zinc sulfate and incubated anaerobically at 37° C. for about 8 hours. After growth was complete, 1% v/v formaldehyde was added for inactivation of the organism. After 7 days, the formaldehyde was partially neutralized with sodium bisulfite. Then the inactivated culture was split into two aliquots. One aliquot was adjuvanted with 15% v/v Carbigen™ while the other was adjuvanted with Carbigen™ at a concentration of 20% v/v.

TABLE I
C. perfringens isolation and test results

| Intestine | | | Alpha | Beta 2 | Neuraminidase |
|---|---|---|---|---|---|
| Sample No. | TpeLsc | NetB | Toxin/mL | Toxin | Activity |
| 1 | + | + | 2400/5600 | + | + |
| 2 | + | + | 3780 | + | + |
| 3 | + | + | 3950 | + | + |
| 4 | + | + | 7490 | + | + |
| 5 | + | + | 2405 | + | + |

### B. Vaccination/Challenge Study Protocol

Newly hatched chicks were separated into ten (10) groups of 30 chicks each. There were five treatments, each comprising two groups of chicks (total equaled 60 chicks per treatment). The chicks were received on Day 0, weighed, vaccinated for New Castle Disease and challenged with Coccidia after which they were treated on the day of hatch (Day 0) with a treatment prepared according to section A above, as described in Table 2.

TABLE 2
Treatment Groups for C. perfringens study

| Treatment | | Formulation |
|---|---|---|
| group | Treatment description | (per dose): |
| 1 | C. perfringens Type A Bacterin | Contains 15% Carbigen ™ |
| | for eye drop application use | |
| | (Hardcrian Gland) | |
| 2 | C. perfringens Type A Bacterin | Contains 20% Carbigen ™ |
| | applied via a hand spray bottle | |
| | to a spot on the bird's back | |
| 3 | C. perfringens Type A Bacterin | Contains 20% Carbigen ™ |
| | for use in Gel Drop Application | mixed 50:50 with a |
| | | commercial gel formu- |
| | | lation and apply to |
| | | chicks topically |
| 4 | C. perfringens Type A Bacterin | Contains 15% Carbigen ™ |
| | for Eye Drop application and a | |
| | second treatment via the choanal | |
| | cleft | |
| 5 | No Treatment (Control group) | NA |

On Day 7, the birds in Treatment Group 4 were administered a booster. All birds were placed on a starter feed from day 0 until day 17 when then were challenged.

On Day 17, all birds were again weighed and challenged with virulent C. perfringens via the feed. The starter feed was weighed back and birds were then placed on grower feed. On Day 21, ten birds per pen were removed and the jejunum tissue was scored for lesions. On Day 28, the birds were again weighed, jejunum tissue samples of all remaining birds were taken and the grower feed was weighed back. Throughout the study, all birds that died were necropsied and the cause of death was noted.

### C. Vaccination/Challenge Study Results

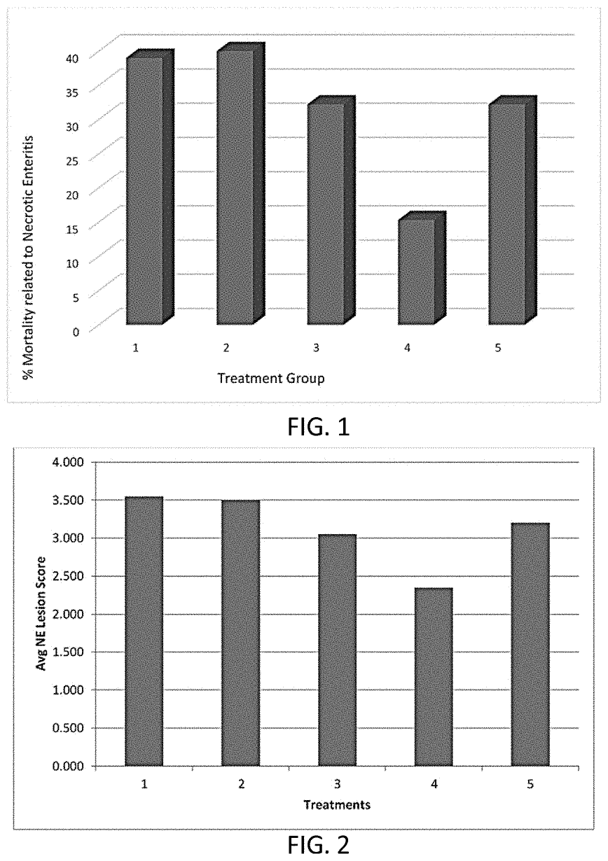

Table 3 and FIGS. 1 and 2 show the mortality and intestinal lesion results for the study. The model used was supposed to produce approximately 15% mortality. However, it was stronger than normal, producing 35% mortality in the controls and up to 40% mortality in some of the other vaccination groups.

TABLE 3
Mortality and intestinal lesion results for each Treatment Group

| | | | | Percent | |
|---|---|---|---|---|---|
| | | | Mortality | Mortality | |
| | No. of | | related to | related to | |
| Treatment | Birds | Total | Necrotic | Necrotic | Intestinal |
| Group | Day 0 | Mortality | Enteritis | Enteritis | Lesion Scores |
| 1 | 30 | 1 | 11 | 38.98 | 3.550 |
| 1 | 30 | 0 | 12 | | |
| 2 | 30 | 0 | 11 | 40.00 | 3.500 |
| 2 | 30 | 0 | 13 | | |
| 3 | 30 | 1 | 9 | 32.20 | 3.050 |
| 3 | 30 | 0 | 10 | | |
| 4 | 30 | 0 | 6 | 15.25 | 2.350 |
| 4 | 30 | 1 | 3 | | |
| 5 | 30 | 2 | 6 | 32.20 | 3.2.00 |
| 5 | 30 | 0 | 13 | | |

Treatment Group 4 demonstrated protection by reducing the mortality related to necrotic enteritis by over 52%, compared to the control Treatment Group 5. All other groups showed no protection compared to the control. Treatment Groups 2 and 3 used a topically-applied procedure that is used for most of the modified live vaccines. With modified live vaccines the organism is live and when the birds peck each other or preen, they get vaccinated with the live organism.

Intestinal lesion analysis also indicated that Treatment Group 4 showed a 26.6% reduction in intestinal lesions whereas none of the other treatment groups showed any change from controls.

The results demonstrated that the disclosed composition comprising inactivated antigens provided protection from necrotic enteritis. Such protection by administering an inactivated vaccine mucosally and then challenging the chicks with a heavy challenge with C. perfringens is a highly significant result because no other vaccine has been shown to reduce necrotic enteritis mortality caused by this organism.

### Example 2

In order to determine whether the results obtained in a research setting would be repeated in the field, the eye drop/chloanal crest application was repeated in a field study at a facility that was experiencing significant problems with necrotic enteritis. 1700 birds were used to ensure that the results would be statistically significant.

In this study, two of the C. perfringens Type A isolates from Example 1 that demonstrated high alpha toxin levels (isolate 4 and isolate 1) were used to prepare a bacterin to use for evaluation in the flock of origin. They were grown in C. perfringens media containing Proteose Peptone, Yeast Extract, Dextrin, Potassium Phosphate monobasic, Lactalbumin Hydrolysate, L-cysteine, N-Z Amine and Deionized water. The pH was adjusted to around 7 with sodium hydroxide. Seed culture was inoculated into vessels containing the media. Isolate 4 was inoculated into a 2 liter vessel while isolate 1 was inoculated into a 1 liter vessel. The vessels were incubated anaerobically for 8 hours at 37° C. after which the cultures were harvested and inactivated with 1% formalin v/v for 7 days at 30-36° C. After inactivation was complete, sodium bisulfite was added to neutralize the formalin and then both cultures were adjuvanted with 20% Carbigen™ with mixing continuing overnight. After mixing was complete, the inactivated and adjuvanted cultures were combined into one serial of bacterin.

The bacterin serial was shipped to the facility from which the isolates were obtained and used to vaccinate 1800 newly hatched chicks using the eye drop/chloanal crest method. In the same house, 22,100 newly hatched chicks from the same hatching were left unvaccinated as controls. The vaccinates were marked with a food-acceptable dye in order to be able to track them for boostering and performance at the end of the study. At day 7, the vaccinated chicks were boostered using the same method as used when newly hatched.

All chicks were monitored with deaths being recorded for each group. The results are shown in Table 4 below. A Chi-square Test was conducted on the data. The results show that there was a statistically significant improvement (P<0.01) in survivability of those that were vaccinated, that survivability being 1.326%. These data clearly demonstrate that the results of the CQR study using inactivated antigens and Carbigen™ adjuvanted vaccines (or bacterins) when applied mucosally can significantly reduce deaths related to C. perfringens that causes necrotic enteritis in young chickens.

TABLE 4
Mortality results at the end of the study

| | No. of | No. of | No. of | Percent |
|---|---|---|---|---|
| Treatment | Chicks | Deaths | Survivors | Deaths |
| Bacterin | 1800 | 61 | 1739 | 3.39 |
| None | 22,100 | 1042 | 21058 | 4.71 |

### Example 3

As described in Example 2, isolates were obtained from dead chickens from a second poultry producer that was losing chickens to necrotic enteritis. The isolates were grown, inactivated and adjuvanted with Carbigen™ at a concentration of 20% as described in Example 2. In the present experiment, the chicks were split into two groups with each group comprising 11,950 birds. One group was vaccinated by placing the chicks into a spray cabinet and spraying them with the inactivated C. perfringens Carbigen-adjuvanted vaccine as disclosed herein. The chicks were passed through the spray cabinet twice to assure that they received an adequate vaccination. These vaccinated chicks were separated by a partition to separate them from the non-vaccinated controls. At 10 days of age, the vaccinates were boostered via spraying with a backpack sprayer. Only total mortality was recorded in each group. The results of this study are shown in Table 5.

TABLE 5
Mortality results at the end of the study

| | No. of | No. of | No. of | Percent |
|---|---|---|---|---|
| Treatment | Chicks | Deaths | Survivors | Deaths |
| Bacterin | 11,950 | 131 | 11,819 | 1.09 |
| None | 11,950 | 283 | 11,667 | 2.37 |

At 17 days of age, 131 of the vaccinated birds had died whereas 283 of the non-vaccinated control birds had died. This was a 54% reduction in deaths. Additionally, the flock supervisor reported that the vaccine appeared to not only protect from necrotic enteritis deaths but also from deaths related to diseases apparently unrelated to necrotic enteritis.

These results indicate that spraying the inactivated, Carbigen-adjuvanted vaccines as disclosed herein onto the chicks using commercial procedures resulted in a significant reduction in deaths.

### Example 4

### Inactivated Salmonella kentucky Sprayed onto Day of Hatch and 14 Day Old Chicks

Salmonella kentucky easily translocates from the intestinal tract to the liver and spleen. Because of this, S. kentucky is the primary Salmonella isolated from broiler processing plants and the most frequent cause for broiler companies to fail USDA performance standards. S. kentucky was used, in part, because it is one of the most difficult Salmonella spp. to prevent from translocating in poultry, and as such, is often used as a test Salmonella species. Typically, if a treatment reduces or substantially prevents S. kentucky translocation, the treatment also will reduce or prevent translocation by other Salmonella spp. in poultry.

The current vaccines on the U.S. market give little to no immunity against S. kentucky. Therefore, the objective of this study is to evaluate whether an inactivated Salmonella vaccine/adjuvant vaccine according to the present disclosure and administered two times by spray application could reduce this translocation or reduce numbers of organisms in the ceca, thus protecting the chickens from a S. kentucky challenge.

In this study, day of age broilers were vaccinated with one (1) dose sprayed of the disclosed vaccine at 1 day (DOT 0) and again at 14 days. At one day of age all birds were tagged with a unique number. On day 30 all birds were co-mingled and orally gavaged with S. kentucky at 10⁷ CFU/chick dose. There were two (2) replicates of each treatment. Each of the two (2) treatments (Table 6) were represented in both isolation rooms which were subdivided into one-half (1/2). Each replicate contained forty-two (42) chicks. The total chicks placed was one-hundred-sixty-eight (168).

TABLE 6
Study design

| | S. kentucky |
|---|---|
| Treatment | Challenge* |

| | | |
|---|---|---|
| 1 | Challenge Control | Yes |
| 2 | Phibro Vaccine Adjuvant at day 1 and 14 | Yes |

*Challenge was on day 30 to insure sufficient time for response to 14 day vaccination.

### Procedure

One-hundred-sixty-eight (168) day-of-hatch Ross×Ross male broiler chicks were obtained from Aviagen Hatchery, Blairsville, Ga. Birds were sexed, received routine vaccinations (HVTSB1), and breeder flock number information was recorded at the hatchery. On DOT 0 all tag numbers were randomly assigned, all chicks were tagged, and tag numbers were recorded by treatments. Samples were taken on days 36 and 43 by tag number as previously determined from randomized table. The vaccines were coarse sprayed at 1 day of age (DOT 0) to Treatment 2 only at one (1) dose per bird in a volume of 0.25 ml per chick. Then on day 14, chicks from treatment 2 were held in a corner of pen and one dose per bird sprayed at 0.25 ml/bird by coarse spray. Sprayer insured coverage of vaccine to beaks and eyes of chicks.

Birds were raised under ambient humidity and were provided a lighting program as per the primary breeder recommendations. At placement, each pen contained approximately four (4) inches of fresh pine shavings. Litter was not replaced during the study course. Each division contained one (1) tube feeder and one (1) bell drinker resulting in a forty (40) bird/feeder and drinker ratio. Feed and watering was ad libitum. All diets contained 113.5 g/ton amprolium. Rations were fed as follows: starter DOT 0 through DOT 22, grower DOT 22 to DOT 30, and finisher DOT 30 to DOT 43. Diets were fed as crumbles (starter feed) or pellets (grower and finisher). Feed formulations for this study consisted of un-medicated commercial-type broiler starter and grower diets compounded with commonly used United States feedstuffs representative of local formulations, calculated analyses to meet or exceed NRC standards. No antibiotics were added to any feed. No concomitant drug therapy was used during the study. To prevent cross-contamination, plastic disposable boots were worn when entering pens and changed between each pen.

Bird weights (kg) by pen were recorded at study initiation (DOT 0), DOT 30 and termination (DOT 43). On DOT 30 all birds were orally dosed (gavaged) with a 0.5 ml dose of approximately 10⁸ CFU/ml for a chick dose of ˜5.0×10⁷ CFU nalidixic acid-resistant Salmonella kentucky.

Sampling by tag number was completed on DOT 36 and DOT 43. On DOT 43 twenty (20) ceca (and not liver/spleen) per group were collected. On DOT 36 twenty (20) birds liver/spleen pool and per replicate (40/treatment) were taken from each individual pen, euthanized (by cervical dislocation), and the pooled liver/spleen by bird or ceca aseptically removed. After removed, each sample was placed in one sterile plastic sample bag (Fisher Scientific), labeled, stored on ice, and transferred to the onsite Southern Poultry Research Group Laboratory for Salmonella analysis. All samples submitted for Salmonella isolation and identification (liver/spleen pool or ceca) were taken to the onsite Southern Poultry Research Group Laboratory on ice in sterile Whirl Pack bags for counting of Salmonella kentucky organisms.

All birds were monitored for general flock condition, temperature, lighting, water, feed, litter condition, and unanticipated house conditions/events. Findings were documented twice daily during the regular working hours (one observation recorded on final study day). One observation recorded Saturday, Sunday, and observed holidays. Pens were checked daily for mortality. Birds were culled only to relieve suffering. The date and removal weight (Kg) was recorded for any bird culled (or found dead), gross necropsy was performed on all culled (or dead) birds, and the following information recorded: gender and probable cause of death.

### Results—Liver/Spleen Salmonella Prevalence

Combined liver/spleen samples were collected from 20 birds per group in each of the two isolation rooms on day 36. Birds from the two treatment groups were comingled in both rooms thus stressing the vaccine. Salmonella prevalence in liver/spleen samples are summarized in Table 7. Thirty-two of 40 birds in the control group were positive whereas only 22 of 40 birds were positive in the vaccinate group. Thus, vaccinated birds had a significantly lower Salmonella prevalence in liver/spleen samples compared to the unvaccinated birds (P=0.010). All Salmonella isolates obtained from liver/spleen samples were identified as belonging to serogroup C2, which was consistent with the S. Kentucky challenge strain.

TABLE 7
Salmonella prevalence (%) in liver/spleen
samples by treatment group

| | No. | No. | |
|---|---|---|---|
| Treatment | Samples | Positive (%) | P |
| Unvaccinated | 40 | 32 (80.0)^{b} | 0.010 |
| Vaccinated | 40 | 22 (55.0)^{a} | |

Percentages with a superscript in common do not differ with a level of significance of 5%.

### Ceca Salmonella Most Probable Number (MPNs)—Culture-Positive Samples

Salmonella MPNs for culture-positive ceca samples are summarized in Table 8. Although there was a reduction in MPN between days 36 and 43 in the vaccinate group, there was no significant effect of treatment (P=0.994), no significant effect of day (P=0.228), and no significant interaction between the effects of treatment and day (P=0.218). If separate comparisons of the treatment groups were performed on each day, there was no significant effect of treatment on either day 36 (P=0.365) or on day 43 (P=0.400).

Two ceca had MPNs that were higher than the other observations in their respective groups: one in the vaccinated group on day 36 and one in the unvaccinated group on day 43. If these two observations were excluded, the mean (SE) of the vaccinated group on day 36 was reduced to 2.36 (0.18) log₁₀ MPN/g and that of the unvaccinated group on day 43 was reduced to 2.13 (0.19) log₁₀ MPN/g. The effects of treatment (P=0.975), day (P=0.199), and the treatment by day interaction (P=0.431), all remained nonsignificant.

TABLE 8
Estimated mean (SE) Salmonella log₁₀ MPN/g in culture-
positive ceca samples by treatment and day

| | | | | Reduction |
|---|---|---|---|---|
| | | | | between Day |
| Treatment | Day 36 | Day 43 | Total | 36 and 43 |
| Unvaccinated | 2.21 (0.19) | 2.22 (0.19) | 2.22^{a} (0.14) | 0 |
| Vaccinated | 2.44 (0.19) | 1.99 (0.19) | 2.22^{a} (0.13) | 0.45 |
| Total | 2.33^{a} (0.13) | 2.10^{a} (0.14) | 2.22 (0.09) | N/A |

Marginal means with a superscript in common do not differ with a level of significance of 5%.

### Performance/Mortality

Feed consumed/(final live weight+mortality weight), and cause of mortality was calculated. The mortality was assessed by gross lesions on necropsy. The results are shown in Tables 9 and 10. There were no significant differences in any performance parameter measured at either 22 days or 30 days. Since both treatments were co-mingled on day 30, final performance differences could not be determined. There was, however, a difference in mortality between the groups. In the control group 3.57% of the birds died whereas in the vaccinate group 2.38% died.

TABLE 9
Day 0 to 22 performance results

| | Feed | | Non- | Weight |
|---|---|---|---|---|
| | Intake | Adjusted | Adjusted | Gain |
| Treatment | (kg/pen) | FCR* | FCR | (kg) |
| 1. Challenge | 46.48A | 1.51A | 1.51A | 0.74A |
| Control | | | | |
| 2. Phibro | 46.35A | 1.53A | 1.54A | 0.73A |
| Vaccine | | | | |

*FCR adjusted for mortality

TABLE 10
Day 0 to 30 performance results

| | Feed | | Non- | Weight | |
|---|---|---|---|---|---|
| | Intake | Adjusted | Adjusted | Gain | Percent |
| Treatment | (kg/pen) | FCR* | FCR | (kg) | Mortality |
| 1. Challenge | 84.70A | 1.48A | 1.50A | 1.40A | 3.57A |
| Control | | | | | |
| 2. Phibro | 83.65A | 1.46A | 1.47A | 1.39A | 2.38A |
| Vaccine | | | | | |

*FCR adjusted for mortality

### Conclusion

The vaccine was very successful in reducing the Salmonella kentucky from translocating from the intestines to internal organs. Since the USDA tests 325 gm of ground chicken which includes these organs, there has been concern that internal organs may be a Salmonella source for positive ground/contaminated poultry. It should also be noted that S. kentucky is highly efficient in colonizing the broilers intestines. The greater numerical decline in MPN/g of ceca content from day 30 to 43 in the vaccinated treatment, as seen in Table 8, may indicate the vaccine was beginning to lower the S.K. numbers in the ceca. Additionally, there was no detrimental effect of the autogenous vaccine on broiler body weight, feed efficiency or livability. Accordingly, vaccination against Salmonella kentucky using the disclosed method and composition can significantly improve meat quality with respect to Salmonella incidence.

### Example 5

In order to determine whether other mucosal routes of administration would effectively protect poultry diseases such as necrotic enteritis, field studies were conducted. In this study a combination of spray application on day three post hatch followed by drinking water application at 10 days of age was evaluated. This study was conducted in 23,900 birds.

Isolates were obtained from dead chickens provided by a producer who was experiencing an outbreak of necrotic enteritis caused by Clostridium perfringens. The isolates were grown, inactivated and adjuvanted with Carbigen™ at a concentration of 20% as described in Example 2.

Twenty-three thousand nine hundred (23,900) chicks housed in a single house were split into two groups with each group comprising 11,950 birds. A divider in the middle of the house, separated the vaccinates from the controls. The control birds were held in the front half of the house and the vaccinates were held in the back half of the same house. At three days of age, the 11,950 vaccinate birds received a spray vaccination using a Stihl backpack sprayer. The vaccine was prepared by adding one gallon of distilled water to 12,250 doses of the vaccine to increase the volume be sufficient for 12,950 doses. Extra doses were prepared because there is loss related to the sprayer during adjustment of the nozzle. The control birds did not receive a treatment. On day 10 the vaccinated birds were boostered via their drinking water. Prior to the vaccine administration, water was withheld for several hours after which they were allowed to drink water containing the vaccine. In this case, 12,250 doses of the vaccine were metered into the drinking water system through a proportioner at the rate of one ounce of vaccine to one gallon of drinking water. The control group had the drinking water withheld for the same period of time but there was no vaccine added to the drinking water that they were allowed to drink. Birds were observed daily for clinical signs of necrotic enteritis.

Clinical necrotic enteritis was observed beginning at day 17 by the flock supervisor. This was consistent with the history in this particular house on this farm. Mortality continued for three consecutive days and began to decrease on the fourth day. The mortality results are shown in table 11. Although the mortality was relatively low, there was a statistically significant difference in the Chi-Square (P=0.0053). Therefore, vaccine administered via spray application on day 3 and oral application on day 10 significantly reduced the incidence of necrotic enteritis.

TABLE 11
Mortality results at the end of the study

| | No. of | No. of | Percent | Chi- |
|---|---|---|---|---|
| Treatment | Chicks | Deaths | Survival | Square |
| Vaccinates | 11,950 | 106 | 99.1013 | 0.0053 |
| None | 11,950 | 69 | 99.4131 | |

These results indicate that spraying followed by oral application of the inactivated, Carbigen-adjuvanted vaccine as disclosed herein, using commercial procedures, resulted in a significant reduction in deaths related to necrotic enteritis.

### Example 6

Although the double spray did not appear to work in the study described in Example 1, it was determined that the spray application was not administered using a commercial-type sprayer. Therefore, a field study was conducted using commercial equipment. In this study a spray application was made on the day of hatch using a commercial spraying chamber. This was followed with a spray applied booster dose administered at 10 days of age. This study was conducted in 33,500 birds.

Isolates were obtained from dead chickens experiencing signs of necrotic enteritis caused by Clostridium perfringens. The isolates were grown, inactivated and adjuvanted with Carbigen™ at a concentration of 20% as described in Example 2. In the present experiment, 33,500 aviagen broiler chicks were split into two groups with each group comprising 16,750 birds. The birds were located in a single house being separated by a divider placed in the middle. The control birds were held in the front half of the house and were not vaccinated. Vaccinates were held in the back half of the same house.

At the first vaccination, a commercial spray cabinet was set up to administer 21 mL of vaccine to each box of 100 chicks. Via this administration, each bird theoretically received about 80% of a dose. A dose would normally be 0.25 mL. The vaccine was administered via this spray cabinet on day of hatch (day 0). At 10 days of age, the vaccinated birds were spray vaccinated using a model SR430 Stihl backpack sprayer with a calculated full dose of 0.25 mL.

All birds were observed for mortality from week 2 through week 5. The mortality results are shown in Table 12. In the control group, there were 373 deaths whereas in the vaccinate group there were 289 deaths. Although there was not a statistically significant difference between the groups, there was a definite decrease in mortality in the vaccinated group. This amounted to a 22.5% reduction in mortality in the vaccinate group when compared to controls. The flock supervisor reported E. coli was present in that house. Therefore, some of the deaths may have been caused by E. coli. This could account for some of the deaths in the vaccinate group.

TABLE 12
Mortality results at the end of the study

| | No. of | No. of | Reduction in |
|---|---|---|---|
| Treatment | Chicks | Deaths | Mortality |
| Vaccinates | 16,750 | 289 | 22.5% |
| None | 16,750 | 373 | N/A |

### Example 7

Because of the low level of necrotic enteritis in the study described in Example 6, a second study evaluating the spray application of inactivated Clostridium perfringens for the reduction in mortality caused by necrotic enteritis was conducted in a second field trial including three poultry houses and 71,700 birds. The protocol was the same as that described in Example 6. Again, birds in the front half of each of the houses were designated controls and birds in the back half of each of the houses were vaccinates. The vaccine was prepared and administered as described in Example 5. Therefore, both vaccine doses were administered by spraying. Each house contained 23,900 birds with 11,950 of the birds in each house being vaccinated and the other 11,950 birds left as non-vaccinated controls.

Birds were observed daily for mortality related to necrotic enteritis during a five week time frame. Once again, necrotic enteritis was minimal during this study. The mortality results for each house are listed in Table 13. House #1 had 45 birds die in the control group and 21 birds die in the vaccinate group. House #2 had 61 birds die of necrotic enteritis in the control group and 42 die of the disease in the vaccinate group. In house #3 there was a difference of 35 deaths in the controls vs 24 deaths in the vaccinates. Although mortality was relatively low during the evaluation period, the mortality in each vaccinate group was decreased as compared with the controls (House #1=53%, House #2=31% and House #3=31% . The mortality in each house was decreased by 31 to 53%, total mortality in the vaccinate groups was reduced by 38% when compared to that in the control groups.

TABLE 13
Mortality results at the end of the study

| | No. of | | | | | |
|---|---|---|---|---|---|---|
| | Chicks in | No. of Deaths | | | Total | Reduction in |
| Treatment | each house | House 1 | House 2 | House 3 | Mortality | Mortality |
| Vaccinates | 11,950 | 21 | 42 | 24 | 87 | 38% |
| None | 11,950 | 45 | 61 | 35 | 141 | N/A |

The results of this example coupled with the results from Example 6 demonstrate that two doses of vaccine applied to birds using commercial spray equipment can reduce mortality caused by necrotic enteritis.

### VI. Exemplary Embodiments

The following numbered paragraphs illustrate exemplary embodiments of the disclosed technology.

Paragraph 1. A composition, comprising inactivated antigens and at least one mucosal adjuvant, the composition formulated for administration to an avian.

Paragraph 2. The composition of paragraph 1, wherein the mucosal adjuvant comprises polyacrylic acid.

Paragraph 3. The composition of paragraph 1 or paragraph 2, wherein the inactivated antigens comprise antigens from one or more of Clostridium perfringens type A, Clostridium perfringens type C, Haemophilus paragalinarum, E. coli spp., Salmonella spp., Pasteurella spp., Campylobacter hepaticus, Avian Chlamydiosis (Chlamydia), Avian Mycoplasma species (Mycoplasma), Avian coccidiosis (Coccidia), reoviruses (REO), avian influenza viruses (IAV-A), Infectious bronchitis virus (IBV), Newcastle Disease Virus (NDV), Fowl Adenovirus (FA), Infectious bursal disease (IBD), Marek's Disease (MDV), Chicken Anaemia (CAV), Infectious Larynogotracheitis (ILTV), Avian Encephalomyelitis (AEV), Avian hepatitis (HEV), Duck hepatitis (DHV), Turkey hemorrhagic enteritis (THEV), or Egg Drop Syndrome virus (EDS).

Paragraph 4. The composition of any one of paragraphs 1-3, wherein the inactivated antigens comprise inactivated antigens from Clostridium perfringens type A.

Paragraph 5. The composition of any one of paragraphs 1-4, wherein the composition further comprises a vehicle, surfactant, inactivating agent, neutralizing agent, cell fragments, or a combination thereof.

Paragraph 6. The composition of paragraph 5, wherein the inactivating agent is formaldehyde, formalin, binary ethyleneimine, thimerosal, beta propiolactone, a detergent, or a combination thereof

Paragraph 7. The composition of any one of paragraphs 1-6, wherein the composition does not comprise saline solution.

Paragraph 8. The composition of any one of paragraphs 1-7, wherein the composition has an osmolarity of from greater than zero to 2% (w/v) sodium chloride solution.

Paragraph 9. The composition of any one of paragraphs 1-8, wherein the composition has a viscosity of from greater than zero to 6 mPa·s. Paragraph 10. The composition of paragraph 9, wherein the viscosity is from 2 mPa·s to 5 mPa·s.

Paragraph 11. The composition of any one of paragraphs 1-10, wherein the adjuvant has an adjuvant concentration in the composition of from greater than zero to 80% (v/v).

Paragraph 12. The composition of paragraph 11, wherein the adjuvant concentration is from 5% to 80%.

Paragraph 13. The composition of paragraph 11, wherein the composition is formulated for addition to drinking water and the adjuvant concentration is from 15% to 80%.

Paragraph 14. The composition of paragraph 11, wherein the composition is formulated for spray administration and the adjuvant concentration is from 15% to 40%. Paragraph 15. The composition of paragraph 11, wherein the composition is formulated for administration to the eye and the adjuvant concentration is from 5% to 25%.

Paragraph 16. The composition of paragraph 11, wherein the composition is formulated for gel administration and the adjuvant concentration is from 15% to 40% prior to mixing with a gel composition. Paragraph 17. A drinking water composition, comprising from greater than zero to 20% (v/v) of the composition of any one of paragraphs 1-13, and water.

Paragraph 18. A gel composition, comprising a first composition comprising the composition of any one of paragraphs 1-12 or 16, and a second composition comprising a gelling agent. Paragraph 19. The gel composition of paragraph 18, wherein a ratio of the first composition to the second composition is from 25:75 to 75:25.

Paragraph 20. The gel composition of paragraph 18, wherein the ratio of the first composition to the second composition is 50:50.

Paragraph 21. A method, comprising administering to an avian the composition of any one of paragraphs 1-20.

Paragraph 22. The method of paragraph 21, wherein administering comprises administering to a mucosal membrane.

Paragraph 23. The method of paragraph 21 or paragraph 22, wherein the avian is a chicken, turkey, goose, duck, Cornish game hen, quail, partridge, pheasant, guinea-fowl, ostrich, emu, swan, or pigeon.

Paragraph 24. The method of paragraph 23, wherein the avian is a chicken or turkey. Paragraph 25. The method of any one of paragraphs 21-24, wherein the composition is administered orally, ocularly or topically.

Paragraph 26. The method of any one of paragraphs 21-25, wherein the composition is administered to an avian of from greater than zero to 14 days of age.

Paragraph 27. The method of any one of paragraphs 21-26, wherein the composition is administered to an avian of from 5 days to 9 days of age.

Paragraph 28. The method of any one of paragraphs 21-24 wherein the composition is administered in ovo.

Paragraph 29. The method of any one of paragraphs 21-28, wherein administering the composition comprises a first administration and a second administration that is subsequent to the first administration.

Paragraph 30. The method of paragraph 29, wherein the first administration occurs when the avian is from greater than zero to 14 days of age.

Paragraph 31. The method of paragraph 29 or paragraph 30, wherein the second administration is from greater than zero to 6 weeks after the first administration. Paragraph 32. The method of paragraph 31, wherein the second administration is from 1 day to 4 weeks after the first administration.

Paragraph 33. The method of paragraph 31, wherein the second administration is from 3 days to 10 days after the first administration.

Paragraph 34. The method of any one of paragraphs 21-33, wherein the composition comprises inactivated Clostridium perfringens type A antigens.

Paragraph 35. The method of any one of paragraphs 21-34, wherein the method comprises administering a composition comprising inactivated Clostridium perfringens type A antigens and a polyacrylic acid adjuvant to an avian of from greater than zero to 14 days of age.

Paragraph 36. The method of any one of paragraphs 21-35, wherein administering to the avian comprises spraying the composition onto the avian.

Paragraph 37. The method of any one of paragraphs 21-35, wherein administering to the avian comprises providing a drinking water composition that comprises the composition to the avian.

Paragraph 38. The method of paragraph 37, wherein the drinking water composition comprises from greater than zero to 20% (v/v) of the composition.

Paragraph 39. The method of paragraph 38, wherein the composition is a composition according to any one of paragraphs 1-13 and the method further comprises mixing the composition and water to form the drinking water composition.

Paragraph 40. The method of any one of paragraphs 21-35, wherein administering to the avian comprises administering the composition to the avian's eye.

Paragraph 41. The method of any one of paragraphs 21-35, wherein administering to the avian comprises providing a gel composition to the avian. Paragraph 42. The method of paragraph 41, wherein the gel composition is a gel composition according to any one of paragraphs 18-20.

Paragraph 43. A method of inducing an immune response in an avian, comprising administrating to the avian the composition of any one of paragraphs 1-20.

Paragraph 44. The method of paragraph 43, wherein inducing an immune response comprises inducing an IgA response in the avian.

Paragraph 45. The method of paragraph 44, wherein inducing an immune response further comprises inducing an IgY response in the avian.

Paragraph 46. A method of treating or preventing necrotic enteritis in an avian, the method comprising administering a composition according to any one of paragraphs 1-20 to the avian, wherein the composition comprises inactivated Clostridium perfringens type A antigens.

In view of the many possible embodiments to which the principles of the disclosed invention may be applied, it should be recognized that the illustrated embodiments are only preferred examples of the technology and should not be taken as limiting the scope of the technology. Rather, the scope of the technology is defined by the following claims. We therefore claim as our invention all that comes within the scope and spirit of these claims.

We claim:

1. A method, comprising mucosally administering a composition comprising inactivated antigens and at least one mucosal, polyacrylic acid-based adjuvant to an avian.

2. The method of claim 1, wherein the composition comprises from 80% to less than 100% water.

3. The method of claim 1, wherein the composition comprises from 0.5% to 50% adjuvant.

4. The method of claim 1, wherein the composition is an aqueous suspension comprising polyacrylic acid particles.

5. The method of claim 1, wherein administering the composition comprises spraying the composition on to the avian.

6. The method of claim 1, wherein administering the composition comprises administering the composition ocularly, nasally and/or orally.

7. The method of claim 1, wherein the inactivated antigens comprise antigens from one or more of Clostridium spp., Haemophilus paragalinarum (Coryza), Escherichia spp., Salmonella spp., Pasteurella spp., Staphylococcus spp., Micrococcus spp., Campylobacter spp., Avibacterium spp., Actinobacillus spp., Neisseria spp., Erysipelothrix spp., Moraxella spp., Avian Chlamydiosis (Chlamydia), Gallibacterium spp., Pseudomonas spp., Rhodococcus spp., Serratia spp., Streptococcus spp., Avian Mycoplasma species (Mycoplasma), Avian coccidiosis (Coccidia), reoviruses (REO), avian influenza viruses (IAV-A), Infectious bronchitis virus (IBV), Newcastle Disease Virus (NDV), Fowl Adenovirus (FA), Infectious bursal disease (IBD), Marek's Disease (MDV), Chicken Anemia (CAV), Infectious Larynogotracheitis (ILTV), Avian Encephalomyelitis (AEV), Avian hepatitis (HEV), Duck hepatitis (DHV), Turkey hemorrhagic enteritis (THEV), or Egg Drop Syndrome virus (EDS).

8. The method of claim 1, wherein the inactivated antigens comprise antigens from Clostridium perfringens type A, Clostridium perfringens type C, Clostridium septicum, Clostridium colinum, E. coli, Salmonella kentucky, Salmonella typhimurium, Salmonella enteriditis, Pasteurella multocida, Pasteurella hemolyca, Campylobacter hepaticus, Campylobacter jejuni, Campylobacter coli, Galibacterium anatis, or a combination thereof.

9. The method of claim 1, wherein the composition further comprises cell fragments, an inactivating agent, a surfactant, neutralizing agent, or a combination thereof

10. The method of claim 1, wherein the composition has:
a viscosity of from 2 mPa·s to 5 mPa·s;
an isotonic osmolarity;
a pH of from 6.5 to 7.5; or
a combination thereof.

11. The method of claim 1, wherein the avian is a chicken, turkey, goose, duck, Cornish game hen, quail, partridge, pheasant, guinea-fowl, ostrich, emu, swan, or pigeon.

12. The method of claim 11, wherein the avian is a chicken or turkey.

13. The method of claim 1, wherein administering the composition comprises administering the composition to the avian on from day of hatch to day 14.

14. The method of claim 13, wherein the composition is administered to the avian on from day of hatch to day 3.

15. The method of claim 1, wherein administering the composition comprises administering a first composition comprising the inactivated antigens and the at least one mucosal, polyacrylic acid-based adjuvant to the avian, and the method further comprises subsequently administering a second composition to the avian.

16. The method of claim 1, wherein the method is a method of treating or preventing necrotic enteritis in an avian.

17. A method for inducing an immune response in an avian, the method comprising mucosally administering an aqueous suspension comprising inactivated antigens, polyacrylic acid particles, and from 80% to less than 100% water to the avian.

18. The method of claim 17, wherein inducing an immune response comprises:
inducing an IgA response in the avian;
inducing an IgY response in the avian; or a combination thereof

19. A method comprising spraying onto an avian, an aqueous suspension comprising:
inactivated antigens selected from antigens from one or more of Clostridium spp., Haemophilus paragalinarum (Coryza), Escherichia spp., Salmonella spp., Pasteurella spp., Staphylococcus spp., Micrococcus spp., Campylobacter spp., Avibacterium spp., Actinobacillus spp., Neisseria spp., Erysipelothrix spp., Moraxella spp., Avian Chlamydiosis (Chlamydia), Gallibacterium spp., Pseudomonas spp., Rhodococcus spp., Serratia spp., Streptococcus spp., Avian Mycoplasma species (Mycoplasma), Avian coccidiosis (Coccidia), reoviruses (REO), avian influenza viruses (IAV-A), Infectious bronchitis virus (IBV), Newcastle Disease Virus (NDV), Fowl Adenovirus (FA), Infectious bursal disease (IBD), Marek's Disease (MDV), Chicken Anemia (CAV), Infectious Larynogotracheitis (ILTV), Avian Encephalomyelitis (AEV), Avian hepatitis (HEV), Duck hepatitis (DHV), Turkey hemorrhagic enteritis (THEV), or Egg Drop Syndrome virus (EDS);
polyacrylic acid particles having a particle size of from 250 nm to 10 microns;
cell fragments, an inactivating agent, a surfactant, neutralizing agent, or a combination thereof; and
from 80% to less than 100% water.

20. The method of claim 19, wherein the inactivated antigens comprise antigens from Clostridium perfringens type A, Clostridium perfringens type C, Clostridium septicum, Clostridium colinum, E. coli, Salmonella kentucky, Salmonella typhimurium, Salmonella enteriditis, Pasteurella multocida, Pasteurella hemolyca, Campylobacter hepaticus, Campylobacter jejuni, Campylobacter coli, Galibacterium anatis, or a combination thereof.
